(19) United States
(12) Patent Application Publication
(10) Pub. No.: US 2018/0008534 A1
(43) Pub. Date: Jan. 11, 2018

(54) METHOD FOR PREVENTING, TREATING, AND CURING CANCER

(71) Applicant: ROBERT B. LOMERSON, FORT WORTH, TX (US)

(72) Inventor: ROBERT B. LOMERSON, FORT WORTH, TX (US)

(21) Appl. No.: 15/534,186

(22) PCT Filed: Dec. 7, 2015
(86) PCT No.: PCT/US15/64335

§ 371 Date: Jun. 8, 2017

Related U.S. Application Data
(60) Provisional application No. 62/089,222, filed on Dec. 8, 2014.

(51) Int. Cl. A61K 9/00; A61K 31/565; A61B 10/02; A61K 31/568; A61M 37/00

(52) U.S. Cl. CPC A61K 9/0024; A61K 31/568; A61K 9/0004; A61M 37/0092; A61M 37/00; A61B 10/0233; A61K 31/565; A61K 9/0046; A61K 9/0009; A61M 2037/0007; A61M 2210/0662

(57) ABSTRACT

The present application includes a method for introducing a hormone directly into an affected area within a tumor. The method includes penetrating the affected area of the tumor with a biopsy needle in order to obtain a biopsy specimen. The specimen is withdrawn along with the biopsy needle. A hormone implant is inserted into the void space left behind as the specimen was removed. The method may also include directly inserting the implant and a treating substance into the tumor without the removal of the biopsy specimen. The implant may have various shapes in order to maximize surface contact. The implant is also configured to have a time release function to regulate dosage delivery.

### TECHNICAL FIELD

The present application relates generally to a medical treatment, and in particular to a method for preventing, treating, and reducing cancer.

### DESCRIPTION OF THE PRIOR ART

Without exception, cancer initiates its appearance within a specific organ such as the prostate in the male and the ovaries and/or breast (for example) in the female. If untreated, cancer may spread or metastasize throughout the body. Thus like ants in the kitchen of a house, it makes little sense to exterminate the whole house rather than just the kitchen where the food is stored. Currently, hormones used to treat low or unbalanced levels of these hormones are generally introduced to the entire body by the use of injections, implanted sub-dermal pills, transdermal patches, creams or gels. Each of these are diluted as they circulate through the entire mass of the body, thereby losing the compounding and force of concentration at the site of interest. Furthermore, this dilution results in an extension of the time required for optimal effect. As a result, this dilution prolongs the development of unwanted side effects. An inability to properly monitor and detect cancer can lead to disastrous consequences. A better method of treatment and regulating hormone levels in the body is needed.

Prostate cancer is unfortunately a common enemy to men as they age. Testing is done to monitor and detect the presence of any potential cancerous tumors. The method of detection and treatment is fairly common. Radiographs are used to determine the number, size, and positions of any tumors. A biopsy is conducted from the locations of potential tumors. A biopsy includes insertion of a hollow needle in to the tumor area. The needle is then removed with a “core sample” inside the needle itself. This is analyzed to determine if there is cancer. From the biopsy, a Gleason score is generated which assesses the severity of the cancer.

Previous methods for the control and elimination of prostate cancer tend to be rather drastic in that they rely on the surgical removal of the prostate gland or the destruction of the tumor within the gland by radiation or cryoablation for example. Each of those processes have associated unwanted lifelong negative side effects typically. Not many other options appear available. Surgery is a method selected by patients in an effort to avoid the effects of radiation. Cryoablation attempts to freeze the cancerous tumor but can inadvertently damage and freeze other adjoining parts of the body at the same time.

Although great strides have been made over the years in medical treatment, an improved method of preventing, or treating cancerous tumors that overcomes the above deficiencies is needed.

### BRIEF DESCRIPTION OF THE DRAWINGS

The novel features believed characteristic of the application are set forth in the appended claims. However, the application itself, as well as a preferred mode of use, and further objectives and advantages thereof, will best be understood by reference to the following detailed description when read in conjunction with the accompanying drawings, wherein:

FIG. 1 is a cross-sectional view of a prostate and a tumor with a biopsy needle introduced to take a core sample according to a preferred embodiment of the present application;

FIG. 2 is a second cross-sectional view of the prostate and tumor of FIG. 1 with the biopsy withdrawn and a treating substance passed through the needle for location within a void of the biopsy sample;

FIG. 3 is a third cross-sectional view of FIGS. 1 and 2 with the needle introducing the treating substance; and

FIGS. 4A-4C are views of various contours and shapes of the treating substance of FIGS. 2 and 3.

While the system and method of the present application is susceptible to various modifications and alternative forms, specific embodiments thereof have been shown by way of example in the drawings and are herein described in detail. It should be understood, however, that the description herein of specific embodiments is not intended to limit the application to the particular embodiment disclosed, but on the contrary, the intention is to cover all modifications, equivalents, and alternatives falling within the spirit and scope of the process of the present application as defined by the appended claims.

### DESCRIPTION OF THE PREFERRED EMBODIMENT

Illustrative embodiments of the preferred embodiment are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming but would nevertheless be a routine undertaking for those of ordinary skill in the art having the benefit of this disclosure.

In the specification, reference may be made to the spatial relationships between various components and to the spatial orientation of various aspects of components as the devices are depicted in the attached drawings. However, as will be recognized by those skilled in the art after a complete reading of the present application, the devices, members, apparatuses, etc. described herein may be positioned in any desired orientation. Thus, the use of terms, such as above and below, to describe a spatial relationship between various components or to describe the spatial orientation of aspects of such components should be understood to describe a relative relationship between the components or a spatial orientation of aspects of such components, respectively, as the device described herein may be oriented in any desired direction.

The method of use in accordance with the present application overcomes one or more of the above-discussed problems commonly associated with existing medical treatment options. Specifically, the method of the present application is configured to provide a less invasive and less harmful option to treat cancer. This method is designed further with the idea to provide minimal risk and efficient treatment of a potentially tumorous area. Maintenance and balancing of a proper estrogen/testosterone level in the body is critical. The method of the present application discusses a method of application of these hormones to a human body and the potential selection of the best implant location. A primary purpose is to cure a patient already having cancer by the use of a hormone or treating substance (i.e. implant) directly within a specific organ, such as the prostate, testicles, uterus, ovaries, etc. Also, a primary purpose is the prevention of the return of the cancer by the use of a balanced hormone implant/treating substance through implantation directly into the organs. These and other unique features of the method are discussed below and illustrated in the accompanying drawings.

The method and device will be understood, both as to its structure and operation, from the accompanying drawings, taken in conjunction with the accompanying description. Several embodiments of the system are presented herein. It should be understood that various components, parts, and features of the different embodiments may be combined together and/or interchanged with one another, all of which are within the scope of the present application, even though not all variations and particular embodiments are shown in the drawings. It should also be understood that the mixing and matching of features, elements, and/or functions between various embodiments is expressly contemplated herein so that one of ordinary skill in the art would appreciate from this disclosure that the features, elements, and/or functions of one embodiment may be incorporated into another embodiment as appropriate, unless otherwise described.

The procedure as outlined in this present application is simple and can be performed on an outpatient basis by any qualified medical personnel. Since this does not require any hospitalization or the associated medical personnel, it is both inexpensive, cost efficient, and can be repeated if required. Perhaps the best of all is that these hormones are normally occurring elements within the human body and thus cannot be rejected or create a problem if the dosage is properly Identified and administered.

The method of treatment in the present application includes the use of a biopsy needle and/or a shaped implant containing a treating substance. The proper selection of an implant location is also included. The treating substance is preferably a biodegradable, time release, estrogen rich material (i.e. estrogen or a balanced hormone risk material). The female hormone, estrogen, has been known to aggressively control and potentially eliminate prostate tumors over time and has been introduced in the past through various delivery methods, including: injection, orally, or transdermally via a patch or gel applied to the surface of the skin. The latter has appeared to be better at affecting the cancer while minimizing the side-effects of taking estrogen.

It is important to note that estrogen can have positive and negative effects on the male body. Estrogen taken orally, by injection, or via transdermal processes typically gets distributed throughout the body and organs resulting in only a portion of the concentrated dosage actually reaching the tumor. In an effort to limit the negative effects of estrogen on the male body, the present application describes a method of providing localized, directed, and potent dosages of estrogen or other treating substances directly to a tumor within the organ itself as opposed to sub-dermally. Furthermore, despite discussion of the disadvantages of oral methods and transdermal applications, it is understood that the method of the present application may include additional steps wherein the treating substance is applied to the body as a whole, as seen in the use of oral methods or transdermal applications. Introduction or application of the treating substance via multiple applications can help to remove, reduce, and prevent cancer. These applications may possibly help restrict metastasis of the cancer.

However, surface skin treatment maintains potential harmful side-effects from the absorption of estrogen into the male system, but that they materialize at a slower rate compared to oral ingestion. It is also important to know that estrogen taken via skin contact tends to circulate throughout the entire body, thereby diluting the dosage that eventually reaches the tumor. Additionally, the time for treatment is lengthened. The method of the present application is configured to accelerate the delivery of the estrogen throughout the tumor while minimizing the side effects by introducing the estrogen treating substance directly into contact with the tumor.

The method of the present application considers two possible routes depending on the situation. First, where a biopsy is needed to determine if a specific location is tumorous, the treating substance may be inserted into the void of the biopsy via the needle. This acts to provide a cautionary dosage and treatment in case the tumor is cancerous. The second method is suited for a situation where the location is known to be cancerous and a biopsy is not needed. In this situation, the method involves the use of the needle (i.e. outer needle and biopsy needle) and/or the direct insertion of the treating substance into the tumorous area via a shaped implant. This treating substance may be formed for providing more surface area in an effort to broaden the surface contact the estrogen has with the tumor in the prostate. It is understood that the use of estrogen in the application is merely an example of a type of treating substance. Other hormones and/or medications may be configured to be delivered in a similar manner. Additionally, although discussion is made in reference to prostate cancer in males, other types of cancer are contemplated with this method. Furthermore, both male and female genders are available to have this method of treatment.

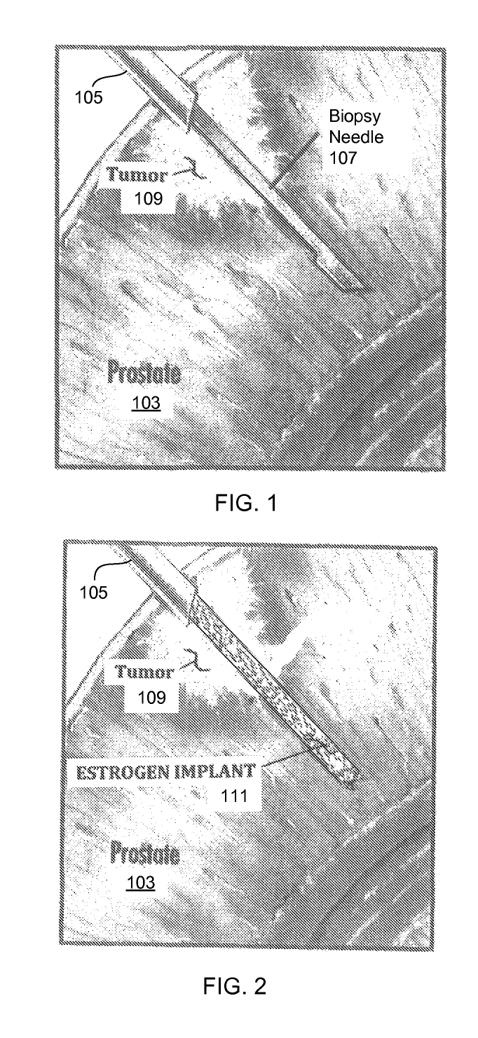

Referring now to FIGS. 1 and 2 in the drawings, the first method of treatment is illustrated. The first method involves the obtaining of a biopsy and then the insertion of the treating substance into the void space left behind by removal of the core sample. In particular with FIG. 1, a cross-sectional view of a prostate 103 having a hypodermic needle 105 enclosing the biopsy needle 107 for obtaining a biopsy is illustrated. The needle 105 and/or 107 is passed through the tumor 109 and preferably into adjoining tissue. The biopsy needle 107 is removed leaving a void space 106. In order to guide the placement of the needle 105 and biopsy needle 107, ultrasound or other guiding methods are utilized. Once the core sample is removed, the void space 106 is at least partially filled with a treating substance or hormone implant 111 (i.e. estrogen implant) that is configured to fill the void space 106. The substance 111 is configured to pass through the tumor 109 (potential tumor) area into the neighboring tissue. Although a tumor 109 is not always confirmed, the insertion of the treating substance is may be made in an effort to act preemptively. This method is naturally also used where the tumor 109 is known to exist but a biopsy may still be needed. By following this method, the treating substance or hormone implant 111 (i.e. estrogen) is placed in direct contact with the tumor 109 directly in the organ, thereby avoiding the slow delivery and small dosages seen through surface skin treatment patches.

The treating substance 111 is typically cylindrical and is configured to be biodegradable and designed to have a time release function so as to regulate the dosage delivery. In the preferred embodiment the treating substance 111 is estrogen rich. It is important to note that the shape and length of the treating substance 111 is not limited to that shown in the Figures. Such features may be modified to accommodate the changing situations and circumstances of the patient prior to delivery within the tissue and tumor 109. Additionally, the treating substance 111 may be other types of hormones or medicine besides estrogen. For example, the treating substance 111 may include a balanced level of estrogen and testosterone in a single implant for treating high-risk patients for cancer or benign prostatic hyperplasia (BPH). This may include the use of a steroid or medicine to decrease the size of the tumor 109. The selected ratio of estrogen and testosterone may be selectively varied between applications of the treating substance 111 to maintain a proper balance. The potency of the treating substance 111 may be varied as well as the time of delivery within the human body depending on circumstances, type, and degree or stage of illness.

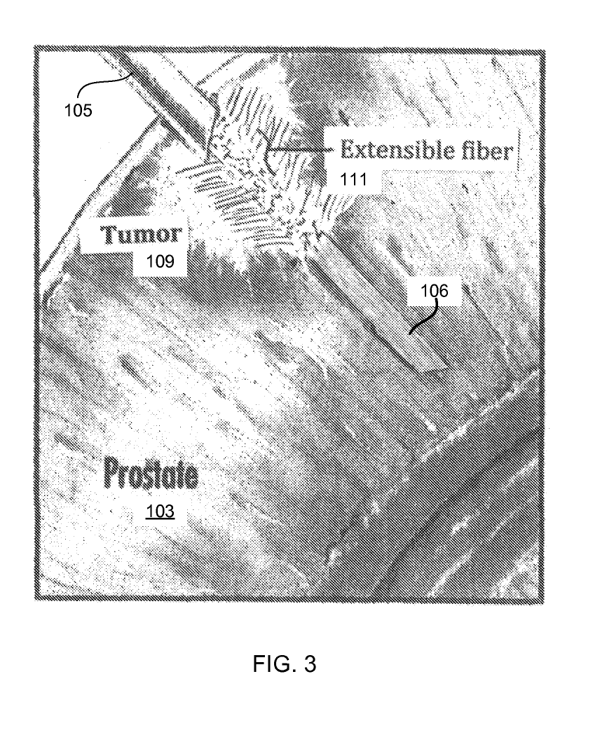

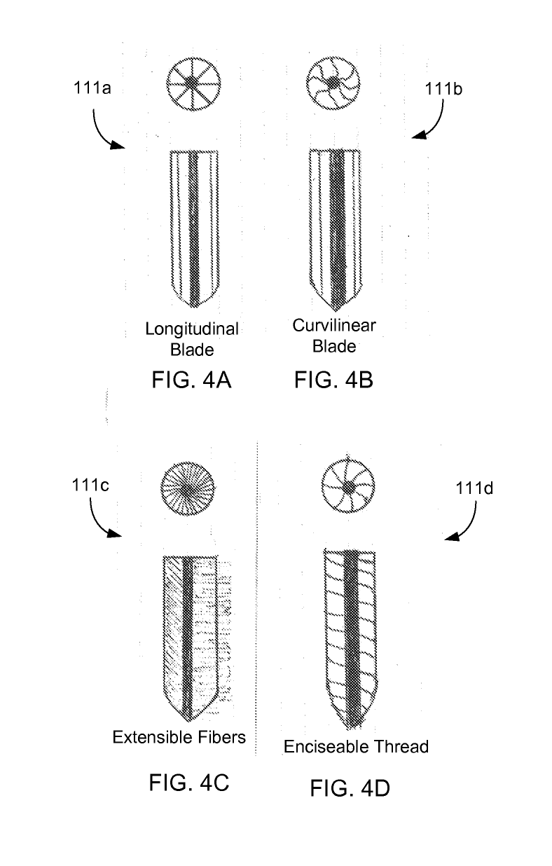

Referring now also to FIG. 3 in the drawings, the second method of treatment is illustrated. FIG. 3 displays the same basic cross-section as shown in FIGS. 1 and 2 except for the step of withdrawing a core sample via the biopsy. A radiograph is used to display the tumor's location, and the treating substance 111 is inserted through the hypodermic needle (outer needle) into direct contact with the tumor 109. As illustrated, the particular “implant” shown is similar to that shown in FIG. 4C described below. The geometry and contours of the treating substance 111 is variable and is configured to increase surface contact with the tumor 109. In this example, extensible fibers are laid-back to simplify the insertion process and upon a slight retraction of the inserting needle, the fibers engage with the tissue and further extend into the tumor 109 until they are perpendicular to the central axis of the implant. In such a position the treating substance 111 obtains a maximum penetration and contact with the tumor 109. Other exemplary shapes are detailed with FIGS. 4A-4D below.

The treating substance 111 of the two methods described herein are in solid form. However, it should be understood that the treating substance 111 is not limited to any specific state (solid or liquid or gas). For example, an injectable gel substance (i.e. a semi-soluable and time released) could be used. Additionally other types of hazardous growths or bodies within a body may be treated apart from tumors or cancer. The two methods contemplate a process that allows and is configured to apply the treating substance 111 in direct contact with the growth, foreign body, tumor, and/or cancer within a body.

Discussion of various implant geometries are provided with respect to FIGS. 4A-4D. The process of inserting the treating substance 111 within the body involves forming an incision or puncturing of the skin wherein an implant is permitted to pass through a portion of the body to reach the tumor 109. The incision may be provided by a needle (i.e. outer needle) or via the implant itself based upon the particular geometry. This is useful when the specific state of the treating substance 111 does not have a solid state. Note that in view of the description below, the implant itself may be configured to be reusable by acting merely as a carrier for the treating substance 111. A single incision may be used for the insertion or reinsertion of one or more implants, thereby minimizing the need to make a plurality of incisions in a localized area of the body.

Referring now also to FIGS. 4A-4D in the figures, various implant geometries of the treating substance 111 are illustrated. A cross sectional view of each geometry is supplied above the side view. A flat blade treating substance might be most obvious in instances where an incision is created, however a single blade may not provide an assurance that the treating substance is uniformly distributed within tumor 109 as desired. Therefore FIG. 4A illustrates a treating substance 111a implant incorporating a series of flat blades radially aligned to that of the central axis. In FIG. 4B, a treating substance 111b implant, shaped as a curvilinear blade is used, wherein the blades are curved as opposed to the linear form seen in FIG. 4A. The implant blade curvatures may be customized to increase the chance of uniform distribution of the treating substance. FIG. 4C illustrates a treating substance 111c having the extensible fibers as discussed in relation to FIG. 3. This may provide the most uniform delivery of the types shown. Each fiber extends out into the tumor 109 or foreign body. Surface contact is maximized. FIG. 4D illustrates a treating substance 111d implant, shaped in threaded form, such that the implant blades encircle the central axis in a continuous radially threaded pattern along a pre-described length. In this embodiment, the implant 111d would be “screwed” into the tumor 109. This shape would work to provide a uniform distribution while at the same time dissecting and interrupting the continuous nature of the tumor 109. Other shapes and embodiments of the treating substance implant blades are contemplated.

In way of further discussion, despite microscopic cancer cells being found in young men in their prime, it has been found that they do not develop into cancer until their 50s or 60s. This profound and overlooked fact provides a specific insight into how cancer can develop and spread; and leads one to question why. The most probable answer lies in the change or alteration of the balance between levels of testosterone and estrogen in the human body.

After consideration of the above facts a hypothesis was developed based on the above information that perhaps a reverse reaction might be capable of destroying and/or eliminating cancer cells/tumor 109s. This hypothesis has been personally verified to be true. Fortunately, or unfortunately depending on how you look at it, the continual use of estrogen patches may not always end as a pleasant experience. Side effects, such as a loss of strength, balance, and an onset of severe back and joint pain may develop and persist from trying to regulate the levels of estrogen/testosterone in the body. Ceasing use of estrogen patches can result in a slight and steady lessening of the side effects and a rebounding of testosterone levels

Since so many adverse health effects occur in both sexes at about 50 to 60 years of age, it makes one wonder just how influential a proper hormone balance (i.e. estrogen/testosterone) is in maintaining a healthy mind and body. Also, could this possibly be remedied to some extent at least by the ongoing maintenance of a proper estrogen/testosterone balance?

Another unexpected event that may occur beyond the noted side effects is a potential for a regrowth of hair in bald scalps. Furthermore, it has been found that hearing may be improved by the repair/regrowth of cilia within the cochlea. The fine hairs are closely related to a new growth of hair on the scalp. It may be possible that a properly balanced and administered estrogen/testosterone hormone level may preserve or restore useful hearing to those who have lost it.

If this hypothesis utilizing a balanced estrogen/testosterone hormone level in conjunction with an interactive feedback loop to the thalamus is correct, the question arises as to where these implants should be the applied. As noted in the present application, implants (primarily estrogen) may be applied directly within the prostate gland since this is where the prostate tumors reside. However other locations are possible depending on the situation. The method of the present application is not limited to an implant location in the prostate.

In view of the above information, that presents a far more encompassing role for the estrogen/testosterone implants as a preventative step as well as a cure. The selection of a site to contain these Implants becomes ever more important. Estrogen and testosterone are perhaps the most basic of all sex hormones since they are intrinsically involved in gender determination and growth, therefore, a potential suitable implant location would possibly be the uterus and/or ovaries for the female with alternate sites as required, and the prostate gland and/or testes be the primary selection sites for the male.

Due to the cochlea's location within the head, the application of a balanced estrogen/testosterone level may require a closer implant location to have a greater affect for hearing especially if the cilia are previously damaged.

This information is meant to further the understanding of the role and effect of the proper balance of the estrogen/testosterone hormones and not to limit the design, implementation or location of these implants. Hopefully this concept can also cover a permanent reusable implant site to facilitate the ongoing reinsertion of the above implants as required.

The current application has many advantages over the prior art including at least the following: (1) localized treatment of the tumor; (2) minimized side effects; and (3) time release function of the treating substance.

It is evident by the foregoing description that the subject application has significant benefits and advantages. The disclosure of the present application is amenable to various changes and modifications without departing from the spirit thereof. The particular embodiments and/or methods disclosed above are illustrative only, as each may be modified and practiced in different but equivalent manners apparent to those skilled in the art having the benefit of the teachings herein. It is therefore evident any alterations, modifications, and all such variations are considered within the scope and spirit of the application. It is apparent that an apparatus with significant advantages has been described and illustrated.

1. A method for introducing a hormone directly into an affected area within a tumor, comprising:
penetrating the affected area of the tumor with a biopsy needle in order to obtain a biopsy specimen;
withdrawing the biopsy needle and a biopsy sample; and
inserting a biodegradable hormone implant into a void space created by the removal of the biopsy sample.

2. The method of claim 1, further comprising:
activating the implant by the use of at least one of an ultrasonic energy, a magnetic vibration, a temperature variation, and a stimulation.

3. The method of claim 1, wherein the implant is shaped as a curvilinear blade to promote uniform distribution of the hormone implant into the tumor.

4. The method of claim 1, wherein the implant is shaped having extensible fibers wherein each fiber extends out into the tumor so as to maximize surface contact in the tumor.

5. The method of claim 1, wherein the implant is shaped in a threaded form, such that the implant threads encircle a central axis in a continuous radial pattern.

6. The method of claim 1, wherein the implant is a biodegradable estrogen rich hormone.

7. The method of claim 1, wherein the hormone implant includes a balance of estrogen and testosterone.

8. The method of claim 1, wherein the hormone implant includes at least one of a steroid and a medicine.

9. The method of claim 1, wherein the hormone implant is configured to have a time release function to regulate dosage delivery.

10. The method of claim 1, wherein the implant is reusable and acts as a carrier of the hormone implant.

11. A method for introducing a treating substance directly into an affected area within a tumor, comprising:
penetrating the affected area of the tumor with a needle; and
inserting an implant into direct contact with the tumor, the implant including the treating substance.

12. The method of claim 11, wherein the implant is shaped as a curvilinear blade to promote uniform distribution of the hormone implant into the tumor.

13. The method of claim 11, wherein the implant is shaped having extensible fibers wherein each fiber extends out into the tumor so as to maximize surface contact in the tumor.

14. The method of claim 11, wherein the implant is shaped in a threaded form, such that the implant threads encircle a central axis in a continuous radial pattern.

15. The method of claim 11, wherein the treating substance is a biodegradable estrogen rich hormone.

16. The method of claim 11, wherein the treating substance includes a balance of estrogen and testosterone.

17. The method of claim 11, wherein the treating substance includes at least one of a steroid and a medicine.

18. The method of claim 11, further comprising:
activating the treating substance by the use of at least one of an ultrasonic energy, a magnetic vibration, a temperature variation, and a stimulation.

19. The method of claim 11, wherein the implant is reusable and acts as a carrier of the treating substance.

20. The method of claim 11, wherein the implant is configured to have a time release function to regulate dosage delivery.

21. The method of claim 11, further comprising:
positioning of the implant with respect to the cochlea, the implant located directly within or in the vicinity of the cochlea.
